(19) United States
(12) Patent Application Publication
(10) Pub. No.: US 2025/0057903 A1
(43) Pub. Date: Feb. 20, 2025

(54) METHOD AND COMPOSITION FOR TREATING NOCTURIA OR IMPROVING SYMPTOMS THEREOF

(71) Applicant: Good Mana LLC, Waimanalo, HI (US)

(72) Inventors: Ian Kitajima, Waipahu, HI (US); Denny Kwock, Honolulu, HI (US)

(73) Assignee: Good Mana LLC, Waimanalo, HI (US)

(21) Appl. No.: 18/773,263

(22) Filed: Jul. 15, 2024

Related U.S. Application Data
(60) Provisional application No. 63/628,724, filed on Aug. 14, 2023.

(51) Int. Cl. A61K 36/185; A23L 33/105; A23L 33/175; A61K 31/198; A61K 36/9066; A61P 13/00

(52) U.S. Cl. CPC A61K 36/185; A23L 33/105; A23L 33/175; A61K 31/198; A61K 36/9066; A61P 13/00; A61K 2236/15

(57) ABSTRACT

Disclosed is a method of treating nocturia or improving symptoms of nocturia in a patient, the method includes administering an effective amount of a composition to the patient, wherein the composition incudes moringa leaves or moringa leaf-prepared substance, and the moringa is Moringa oleifera. Also disclosed is a dietary supplement containing ground moringa leaf powder, and the ground moringa leaf powder is at an amount of 100 mg to 3000 mg per daily dose.

### CROSS-REFERENCING

This application claims the benefit of provisional application No. 63/628,724, filed on Aug. 14, 2023, which are all hereby incorporated in reference by their entireties for all purposes.

### FIELD OF THE INVENTION

The invention relates to a method of treating a sleep disorder. More specifically, it relates to a method of treating nocturia or improving symptoms of nocturia with an extract of moringa, and methods of using the prepared an extract of moringa.

### BACKGROUND OF THE INVENTION

Sleep is foundational to good health. Consistent, quality sleep reduces the risk for strokes, heart attacks, diabetes, and other health issues. Good sleep is also essential for mental clarity, productivity, and emotional well-being.

Unfortunately, the urge to frequently urinate at night, nocturia, is highly disruptive to a good night's sleep. Besides, getting up multiple times at night increases the risk of falls, especially in older adults. This can lead to injuries such as fractures or head injuries, which can have significant health implications. Furthermore, chronic sleep deprivation due to nocturia can affect daily activities, work performance, and overall well-being. It can lead to decreased productivity, increased stress, and a lower quality of life.

There are various underlying causes for nocturia including age-related change in anti-diuretic hormone (arginine, citrulline, and/or beet root extract-vasopressin) diurnal patterns, venous insufficiencies, age-related changes in urinary tract structure and function including benign prostatic hyperplasia (BPH) in men, and various underlying medical conditions including diabetes and kidney disease. An individual may suffer from nocturia as a result of having one or more of these conditions.

There are various dietary supplements and nutraceutical formulas on the market that address some of these individual conditions, but there is no known related art showing the application of the exact or similar dietary supplement formulation described herein to address nocturia.

For example, there are various dietary supplements on the market that address benign prostatic hyperplasia (BPH) in men containing ingredients such as saw palmetto, pumpkin seed oil, pygeum bark, and cranberry seed extract. Some of these same ingredients are also used to help on overactive bladder which can contribute to nocturia in both men and women. An overactive bladder or obstruction in the urinary tract from BPH in men is not specifically a night time issue or always present in those suffering from nocturia. Moreover, none of these related art ingredients are known to have significant impact on the broad range of underlying causes for nocturia.

Clearly, there is an urgent and strong need to develop a treatment agent for nocturia or improving symptoms related to nocturia in a person. Managing nocturia effectively can significantly improve a person's sleep quality, overall health, and daily functioning.

### SUMMARY OF THE INVENTION

This invention describes the use of dietary supplement formulation to treat nocturia. More specifically, the dietary supplement formulation is prepared from plant Moringa oleifera. Moringa oleifera is a fast-growing, drought-resistant tree of the family Moringaceae, native to the Indian subcontinent and used extensively in South and Southeast Asia.

This invention provides a method as well as a dietary supplement as outlined below. The method largely relies on organic agents, unexpectedly, delivers an effective solution. In conducted studies, it reduced the severity of nocturia for patients and, in some instance, it all but eliminated the symptoms of nocturia.

One aspect of this invention relates to a method of treating nocturia or improving symptoms of nocturia in a patient. The method includes administering an effective amount of a composition to the patient, and the composition contains moringa leaves or moringa leaf-prepared substance.

The moringa can be Moringa oleifera, Moringa stenopetala, or a combination thereof. Specifically, the moringa explored in this invention is Moringa oleifera.

Preferably, the composition of this invention comprises ground moringa leaf powder.

As the treatment method targets nocturia, the administering to the patient is, advantageously, one hour before the bedtime quaque die and at a daily dose of 100 mg to 3000 mg of the ground moringa leaf powder.

Preferably, the daily dose for the ground moringa leaf powder can be 500 mg to 1000 mg.

In one embodiment of the method, the daily dose for the ground Moringa oleifera leaf powder is 500 mg to 1000 mg. More specifically, the daily dose for the ground Moringa oleifera leaf powder is 500 mg.

In another embodiment, the composition further comprises an antioxidant and anti-inflammatory agent at an amount of 100 mg to 3000 mg per daily dose. One example of the antioxidant and anti-inflammatory agent is turmeric. Under the example, the daily doses for the ground Moringa oleifera leaf powder and the turmeric can be 500 mg and 500 mg, respectively.

In yet another embodiment, the composition further comprises a circulation enhancer at an amount of 100 mg to 3000 mg and an antioxidant and anti-inflammatory agent at an amount of 100 mg to 3000 mg per daily dose. The circulation enhancer can be arginine, citrulline, or beet root extract and the antioxidant and anti-inflammatory agent can be turmeric. More specifically, the circulation enhancer is arginine. Thus, one example of the composition contains ground Moringa oleifera leaf powder, turmeric, and arginine. Particularly for the above method, the daily doses for the ground Moringa oleifera leaf powder, the turmeric, and the arginine are 500 mg, 500 mg, and 1000 mg, respectively.

For the above treatment method, the composition can be in a solid, semi-solid, or liquid form. As drugs can be formulated into various forms to suit different needs and preferences, the composition of this invention can be formulated in forms that improve efficacy and enhance patient compliance.

Examples of suitable forms of the composition include tablet, which can be extended-release, chewable, or orally disintegrating type, capsule, or liquid, which can be solutions or suspensions.

In the method described above, the administering the composition can be by orally, buccally, rectally, parenterally, intravenously, intraperitoneally, intradermal, subcutaneously, intramuscularly, trans-dermally, or intratracheally.

Another aspect of this invention relates to a dietary supplement for treating nocturia or improving symptoms thereof. The dietary supplement contains ground moringa leaf powder at an amount of 100 mg to 3000 mg per daily dose.

Examples of the moringa, as discussed above, can be Moringa oleifera, Moringa stenopetala, or a combination thereof. Specifically, the moringa is Moringa oleifera.

In one embodiment of the dietary supplement, the ground Moringa oleifera leaf powder is at the amount of 500 mg to 1000 mg per daily dose. Preferably, the ground Moringa oleifera leaf powder is at the amount of 500 mg.

In another embodiment, the dietary supplement contains an antioxidant and anti-inflammatory agent at an amount of 100 mg to 3000 mg per daily dose. As pointed out above, the antioxidant and anti-inflammatory agent can be turmeric. Thus, the dietary supplement can contain the ground Moringa oleifera leaf powder and the turmeric at the amount of 500 mg and 500 mg, respectively.

In still another embodiment, the dietary supplement contains a circulation enhancer at an amount of 100 mg to 3000 mg and an antioxidant and anti-inflammatory agent at an amount of 100 mg to 3000 mg per daily dose. The circulation enhancer can be arginine, citrulline, or beet root extract and the antioxidant and anti-inflammatory agent can be turmeric. More specifically, the circulation enhancer is arginine. Thus, the dietary supplement for daily dose of this invention can contain the ground Moringa oleifera leaf powder, the turmeric, and the arginine at the amount of 500 mg, 500 mg, and 1000 mg, respectively.

Yet another aspect of this invention relates to a method of manufacturing the dietary supplement described above. The manufacturing can be in a capsule format for ease of administration and consistent dosing, or can be in a tablet format incorporating excipients to achieve a solubility rate that optimizes blood plasma level for a long duration to promote efficacy.

The details of the invention are set forth in the drawing and description below. Other features, objects, and advantages of the invention will be apparent from the description and from the claims.

### DETAILED DESCRIPTION

Before the present methods are described, it is to be understood that this invention is not limited to particular method described, as such may, of course, vary. It is also to be understood that the terminology used herein is for the purpose of describing particular embodiments only, and is not intended to be limiting, since the scope of the present invention will be limited only by the appended claims.

Nocturia is common in adults 50 years and older and has a number of underlying root causes. In accordance with the present invention, novel dietary supplement compositions are provided that reduces or eliminates nocturia, the need for frequent urination during the night that interferes with sleep. Described also is a method for achieving said benefits with said compositions and multiple ways these compositions can be prepared.

There are multiple health and age-related factors that result in nocturia. Described is a novel approach to use moringa or compositions that include moringa to induce physiological changes that reduce or eliminate the occurrence of nocturia. Beneficial physiological changes that reduce the occurrence of nocturia include normalizing anti-diuretic hormone (arginine, citrulline, and/or beet root extract-vasopressin) diurnal patterns, improving glucose metabolism, improving vascularity, inflammation reduction and reduction of free radicals that can irritate the bladder and urinary tract. These beneficial physiological changes are achieved with moringa alone or moringa combined with other ingredients to achieve synergistic benefits not realized with individual ingredients. Possible synergistic effects include multiple mechanisms of action to addressing the underlying causes of nocturia and achievement of results at a lower dose than possible with the individual ingredients of the composition. Initial data shows that the described invention can be effective with a single dose taken one hour before bedtime without the need to take the described composition for several days or weeks before being effective.

The method and dietary supplement prepared as described in this invention can be used for therapeutic purposes: treating nocturia or improving symptoms related to nocturia in a human subject.

The subject methods are useful primarily for therapeutic purposes. Thus, as used herein, the term “treating” is used to refer to both prevention of disease, and treatment of a pre-existing condition. The treatment of ongoing disease, to stabilize or improve the clinical symptoms of the patient, is a particularly important benefit provided by the present invention. Such treatment is desirably performed prior to loss of function in the affected tissues including the cardiovascular system and its surrounding tissues. For example, treatment of a patient suffering from a sleep disorder may treating related health issues such as sleep apnea, restless legs syndrome, or chronic pain that may contribute to sleep disturbances.

The terms “improving,” “inhibiting,” “reducing,” or “prevention,” or any variation of these terms, when used in the claims and/or the specification includes any measurable decrease or complete inhibition to achieve a desired result.

On the other hand, the terms “determining,” “measuring,” and “assessing,” and “assaying” are used interchangeably and include both quantitative and qualitative determinations.

The terms “subject,” “host,” “patient,” and “individual” are used interchangeably herein to refer to any mammalian subject for whom diagnosis or therapy is desired, particularly humans. Other subjects may include cattle, dogs, cats, guinea pigs, rabbits, rats, mice, horses, and so on.

The terms “antioxidant” is used for molecules that inhibit the oxidation of other molecules. Oxidation is a chemical reaction that can produce free radicals, which are unstable atoms that can cause cell damage. Antioxidants neutralize free radicals, preventing them from causing harm to cells and tissues. Antioxidants are found in a variety of foods, particularly fruits and vegetables. Common antioxidants include vitamins C and E, selenium, and flavonoids.

The terms “anti-inflammatory agent” is used for molecules that reduce inflammation in the body, which is a natural response to injury or infection but can lead to chronic conditions if it persists. These agents can be found in certain foods, medications, and supplements. They work by inhibiting the pathways that lead to inflammation, thereby reducing symptoms such as pain, swelling, and redness. Examples of anti-inflammatory agent include Non-Steroidal Anti-Inflammatory Drugs (NSAIDs), such as ibuprofen, aspirin, and naproxen. These drugs inhibit the enzyme cyclooxygenase, which is involved in the production of pro-inflammatory chemicals called prostaglandins.

Some substances exhibit both antioxidant and anti-inflammatory properties, offering dual benefits. For example, turmeric (also known as curcumin) is both a powerful antioxidant and an anti-inflammatory agent.

A circulation enhancer is a substance or practice that improves blood flow throughout the body. Enhanced circulation ensures that oxygen and nutrients are efficiently delivered to tissues and organs, while waste products are effectively removed. Improved circulation can have various health benefits, including increased energy, reduced risk of cardiovascular diseases, and better overall health. Types of circulation enhancers include medicinal drugs such as vasodilators that relax blood vessel walls, allowing blood to flow more easily, and anticoagulants, also known as blood thinners that prevent blood clots from forming.

In the dietary supplement of this invention, the ground Moringa oleifera leaf powder can be at the amount of 200 mg to 3000 mg, 200 mg to 500 mg, 400 mg to 500 mg, 500 mg to 3000 mg, 500 mg to 2000 mg, 500 mg to 1000 mg, 500 mg to 800 mg, 800 mg to 1000 mg, 1000 mg to 1500 mg, 1000 mg to 2000 mg, 1500 mg to 2000 mg, 2000 mg to 2500 mg, or 2000 mg to 3000 mg per daily dose.

An “effective amount” is an amount sufficient to effect beneficial or desired clinical results. An effective amount can be administered in one or more administrations. For purposes of this invention, an effective amount of reagent antibodies is an amount that is sufficient to diagnose, palliate, ameliorate, stabilize, reverse, slow or delay the progression of the disease state.

Bioavailability refers to the extent and rate at which the active moiety (drug or metabolite) enters systemic circulation, thereby accessing the site of action. Bioavailability for a given formulation provides an estimate of the relative fraction of the orally administered dose that is absorbed into the systemic circulation. Low bioavailability is most common with oral dosage forms of poorly water-soluble, slowly absorbed drugs. Insufficient time for absorption in the gastrointestinal tract is a common cause of low bioavailability. If the drug does not dissolve readily or cannot penetrate the epithelial membrane (e.g., if it is highly ionized and polar), time at the absorption site may be insufficient. Orally administered drugs must pass through the intestinal wall and then the portal circulation to the liver, both of which are common sites of first-pass metabolism (metabolism that occurs before a drug reaches systemic circulation). Thus, many drugs may be metabolized before adequate plasma concentrations are reached.

Actual dosage levels of the active ingredients in the presently disclosed compositions can be varied so as to obtain an amount of the active ingredient that is effective to achieve the desired therapeutic response for a particular subject, composition, route of administration, and disease, disorder, or condition without being toxic to the subject. The selected dosage level will depend on a variety of factors including the activity of the particular composition employed, the route of administration, the time of administration, the rate of excretion of the particular composition being employed, the duration of the treatment, other drugs, and/or materials used in combination with the particular composition employed, the age, sex, weight, condition, general health and prior medical history of the patient being treated, and like factors well known in the medical arts.

A physician having ordinary skill in the art can readily determine and prescribe the effective amount of the presently disclosed composition required. Accordingly, the dosage range for administration may be adjusted by the physician as necessary, as described more fully elsewhere herein.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art.

### BEST MODE FOR CARRYING OUT INVENTION

The following example explains the present invention more concretely, but do not limit the range of the present invention.

### EXAMPLES

### Example 1: Preparation of Ground Moringa oleifera Leaf Powder

Moringa oleifera is a fast-growing tree in tropical climates. The leaves are harvested and dried with moderate heat to reduce moisture content to below 4%. The leaves are than ground to a 60-80 mesh to create a powder that are encapsulated in the appropriate size edible capsules for consistent dosing and easy consumption. The powder is used in itself or incorporated with additional ingredients in the following formulations:

| | INGREDIENT COMPONENTS OF EFFECTIVE DOSE RANGES | | |
|---|---|---|---|
| | | Antioxidant/Anti- | Circulation Enhancer |
| | | inflammatory | (arginine, citrulline, and/or |
| | Moringa oleifera | (turmeric) | beet root extract) |
| Formulation 1 | 100 mg to 3000 mg | | |
| Formulation 2 | 100 mg to 3000 mg | 100 mg to 3000 mg | |
| Formulation 3 | 100 mg to 3000 mg | 100 mg to 3000 mg | 100 mg to 3000 mg |

Delivery forms of the above formulations include tablet or caplet. They may include excipients to optimize solubility and blood plasma level. The formulation was taken one hour before bedtime on a daily basis.

Moringa oleifera leaf extract is potentially toxic at levels exceeding 3,000 mg/kg of body weight, but safe at levels below 1,000 mg/kg. Moringa oleifera may interfere with prescription drugs affecting cytochrome P450 (including CYP3A4) and may inhibit the antihyperglycemic effect of sitagliptin.

### Example 2: Evaluation of Effects of the Dietary Supplement of this Invention

Experiment: The described formulations were provided to 9 male adults in their mid-50s or older who reported the need to regularly wake at night to urinate. They were asked to report how many times they would typically wake to urinate as a baseline. They were instructed to take one capsule of the formulation 1 hour before bedtime for 5 nights and to log how many times they needed to wake to urinate each of those nights along with number of hours slept.

Statistical Analysis: A one-tailed p-value analysis was performed on the frequency of nightly urination prior to treatment versus during the 5 nights of treatment resulting in a p-value of 0.0000705.

Mathematical statistical analysis was performed. A p-value of less than 0.01 (p<0.01) was considered to indicate statistical significance.

The Results from the above experiment is shown in Table below:

TREATMENT DATA FOR NINE (9) MEN OVER 5 NIGHTS ON NOCTURIA DIETARY SUPPLEMENT FORMULATIONS

| | | AVG | | | | | | | | AVG |
|---|---|---|---|---|---|---|---|---|---|---|
| | | NIGHTLY | | | FREQUENCY NIGHT | | | | | NIGHTLY |
| | | URINATION | TREATMENT | | URINATION/HOURS SLEPT | | | | | URINATION |
| PATIENT | | PRE- | START | | NIGHT | NIGHT | NIGHT | NIGHT | NIGHT | DURING |
| ID | AGE | TREATMENT | DATE | FORMULATION | 1 | 2 | 3 | 4 | 5 | TREATMENT |
| IK | 59 | 3.0 | May 21, 2023 | 1 | 0/7 | 0/6.5 | 1/7.5 | 0/7 | 0/5 | 0.2 |
| KM | 61 | 3.5 | May 21, 2023 | 1 | 2/8 | 1/7.5 | 0/6 | 1/8 | 1/7.5 | 1.0 |
| DK | 57 | 1.0 | May 28, 2023 | 1 | 0/4.5 | 0/6.5 | 0/6 | 0/7 | 0/6.5 | 0.0 |
| EE | 58 | 2.5 | Jun. 4, 2023 | 1 | 1/6 | 1/5 | 0/5 | 2/7 | 1/8 | 1.0 |
| CK | 53 | 1.0 | Jun. 4, 2023 | 2 | 1/8 | 0/7.5 | 0/7.5 | 1/8.5 | 0/7.5 | 0.4 |
| OY | 63 | 2.5 | Jun. 4, 2023 | 2 | 1/7 | 0/7 | 1/7 | 0/7 | 1/9 | 0.6 |
| MN | 61 | 3.0 | Jun. 4, 2023 | 2 | 1/7.5 | 1/8 | 0/6 | 1/8 | 1/7.5 | 0.8 |
| RW | 65 | 3.5 | Jun. 11, 2023 | 3 | 2/7.5 | 1/7.5 | 0/7.5 | 1/7.5 | 1/7.5 | 1.0 |
| TI | 75 | 4.5 | Jun. 11, 2023 | 3 | 1/6 | 2/7 | 1/7 | 0/5 | 1/7 | 1.0 |
| | | | | | | | | | p-value: | 0.0000705 |

| | |
|---|---|
| FORMULATION 1: | 500 mg moringa |
| FORMULATION 2: | 500 mg moringa + 500 mg turmeric |
| FORMULATION 3: | 500 mg moringa + 500 mg turmeric + 1000 mg orginine |

Taken one hour before bedtime.

The results show that compared with pre-treatment condition, the formulations according to this invention has a significantly reduced frequencies of nightly urination for tested patients, demonstrating a therapeutic effect on nocturia.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

In the event that any patents, patent applications, or other references are incorporated by reference herein and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

It is believed that the following claims particularly point out certain combinations and subcombinations that are directed to one of the disclosed inventions and are novel and nonobvious. Inventions embodied in other combinations and subcombinations of features, functions, elements and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

What is claimed is:

1. A method of treating nocturia or improving symptoms of nocturia in a patient, comprising administering an effective amount of a composition to the patient, wherein the composition comprises moringa leaves or moringa leaf-prepared substance.

2. The method according to claim 1, wherein the moringa is Moringa oleifera.

3. The method according to claim 1, wherein the moringa is Moringa oleifera, Moringa stenopetala, or a combination thereof.

4. The method according to claim 1, wherein the composition comprises ground moringa leaf powder.

5. The method according to claim 4, wherein the administering to the patient is one hour before the bedtime quaque die and at a daily dose of 100 mg to 3000 mg of the ground moringa leaf powder.

6. The method according to claim 5, wherein the daily dose for the ground moringa leaf powder is 500 mg to 1000 mg.

7. The method according to claim 5, wherein the composition further comprises an antioxidant and anti-inflammatory agent at an amount of 100 mg to 3000 mg per daily dose.

8. The method according to claim 7, wherein the antioxidant and anti-inflammatory agent is turmeric.

9. The method according to claim 7, wherein the composition further comprises a circulation enhancer at an amount of 100 mg to 3000 mg per daily dose.

10. The method according to claim 9, wherein the circulation enhancer is arginine, citrulline, or beet root extract.

11. The method according to claim 9, wherein the composition is in a solid, semi-solid, or liquid form and the administering the composition can be by orally, buccally, rectally, parenterally, intravenously, intraperitoneally, intradermal, subcutaneously, intramuscularly, trans-dermally, or intratracheally.

12. The method according to claim 6, wherein the moringa is Moringa oleifera and the daily dose for the ground Moringa oleifera leaf powder is 500 mg to 1000 mg.

13. The method according to claim 12, wherein the daily doses for the ground Moringa oleifera leaf powder and the turmeric are 500 mg and 500 mg, respectively.

14. The method according to claim 9, wherein the moringa is Moringa oleifera and the daily dose for the ground Moringa oleifera leaf powder is 500 mg to 1000 mg.

15. The method according to claim 14, wherein the antioxidant and anti-inflammatory agent is turmeric and the circulation enhancer is arginine.

16. The method according to claim 15, wherein the daily doses for the ground Moringa oleifera leaf powder, the turmeric, and the arginine are 500 mg, 500 mg, and 1000 mg, respectively.

17. A dietary supplement for treating nocturia or improving symptoms thereof, comprising ground moringa leaf powder at an amount of 100 mg to 3000 mg per daily dose.

18. The dietary supplement according to claim 17, wherein the moringa is Moringa oleifera and the ground Moringa oleifera leaf powder is at an amount of 500 mg per daily dose.

19. The dietary supplement according to claim 18, further comprising turmeric at an amount of 500 mg per daily dose.

20. The dietary supplement according to claim 19, further comprising arginine at an amount of 1000 mg per daily dose.
